(19) United States
(12) Patent Application Publication
(10) Pub. No.: US 2026/0325138 A1
(43) Pub. Date: Oct. 22, 2026

(54) SURGICAL METHOD FOR TREATING AORTIC DISSECTION

(71) Applicant: AORVEN MEDICAL, INC., Costa Mesa, CA (US)

(72) Inventor: Dai Yamanouchi, Madison, WI (US)

(73) Assignee: AORVEN MEDICAL, INC., Costa Mesa, CA (US)

(21) Appl. No.: 19/650,291

(22) Filed: Apr. 16, 2026

Related U.S. Application Data
(60) Provisional application No. 63/791,171, filed on Apr. 18, 2025.

(51) Int. Cl. A61B 17/11

(52) U.S. Cl. CPC A61B 17/11; A61B 2017/1107; A61B 2017/1139

(57) ABSTRACT

A surgical method for treating aortic dissection includes advancing a penetration device along a vein to a position in the vein adjacent a false lumen of an aorta, advancing a wire at a distal end of the penetration device from the vein into the false lumen of the aorta, establishing a fluid connection between the vein and the false lumen by advancing a shunt device along the wire to a position connecting the vein with the false lumen, and causing, by the fluid connection, depressurization and collapse of the false lumen thereby treating the aortic dissection.

### CROSS-REFERENCE TO RELATED APPLICATION

This application claims the benefit of and priority to U.S. Provisional Application No. 63/791,171, filed Apr. 18, 2025, the entire disclosure of which is incorporated by reference herein.

### BACKGROUND

The present disclosure relates generally to systems and methods for treating diseased blood vessels, and more particularly to surgical devices and techniques for treating diseased vessels.

### SUMMARY

One embodiment relates to a surgical method for treating aortic dissection. The method includes advancing a penetration device along a vein to a position in the vein adjacent a false lumen of an aorta, advancing a wire at a distal end of the penetration device from the vein into the false lumen of the aorta, establishing a fluid connection between the vein and the false lumen by advancing a shunt device along the wire to a position connecting the vein with the false lumen, and causing, by the fluid connection, depressurization and collapse of the false lumen thereby treating the aortic dissection.

Another embodiment relates to a surgical method for treating aortic dissection. The method includes advancing a device along a wire to be positioned between a vein and a false lumen of an aorta, expanding the device to establish a fluid connection between the vein and the false lumen, causing, by the fluid connection, depressurization of the false lumen thereby treating the aortic dissection, and removing the device responsive to the depressurization of the false lumen.

Yet another embodiment relates to a system for treating aortic dissection. The system includes a penetration device configured to advance along a vein to a position in the vein adjacent a false lumen of an aorta and a shunt device configured to advance along the wire and establish a fluid connection between the vein and the false lumen in a deployed configuration. The penetration device includes a wire at a distal end thereof. The wire is configured to extend into the false lumen. The shunt device includes a first radially expanding portion, a second radially expanding portion, and a connecting portion positioned between the first and second radially expanding portions. The fluid connection causes depressurization and collapse of the false lumen, thereby treating the aortic dissection.

### BRIEF DESCRIPTION OF THE DRAWINGS

FIG. 1 is an illustration of an aorta, according to an exemplary embodiment.

FIG. 2 is an illustration of an aorta dissection of the aorta of FIG. 1, according to an exemplary embodiment.

FIG. 3 is a flowchart of a process for treating an aortic dissection, according to some embodiments.

FIG. 4 is a first illustration of a step from the process of FIG. 3, according to an exemplary embodiment.

FIG. 5 is a second illustration of a step from the process of FIG. 3, according to an exemplary embodiment.

FIG. 6 is a third illustration of a step from the process of FIG. 3, according to an exemplary embodiment.

FIG. 7 is a fourth illustration of a step from the process of FIG. 3, according to an exemplary embodiment.

FIG. 8 is a fifth illustration of a step from the process of FIG. 3, according to an exemplary embodiment.

FIG. 9 is a sixth illustration of a step from the process of FIG. 3, according to an exemplary embodiment.

FIG. 10 is a seventh illustration of a step from the process of FIG. 3, according to an exemplary embodiment.

### DETAILED DESCRIPTION

Presently preferred embodiments of the invention are illustrated in the drawings. An effort has been made to use the same or like reference numbers throughout the drawings to refer to the same or like parts. Although this specification refers primarily to methods for treating aortic dissections, it should be understood that the subject matter described herein is applicable to other vessels of the body, and/or to other types of medical systems, including those used for surgical and non-surgical applications.

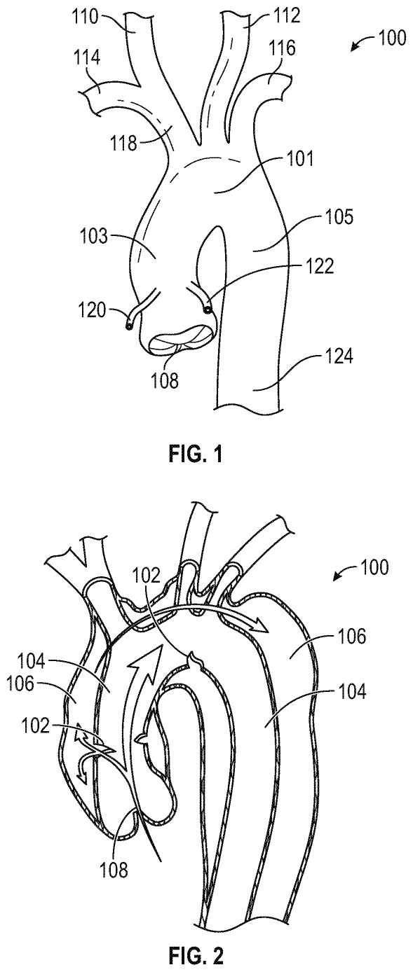

FIG. 1 illustrates an example of an aorta 100. The aorta includes an aortic arch 101, an ascending aorta 103, a descending aorta 105, and an aortic valve 108. The aorta is the main artery that provides oxygenated blood from the heart to the rest of the body and primarily functions to distribute the oxygenated blood to various organs and tissues. The left ventricle of the heart pumps blood from the left ventricle through the aortic valve 108 into the aorta 100. The aorta 100 includes various arteries that fluidly couple the aorta 100 to various regions of the body. These arteries include, but are not limited to, a right common carotid artery 110 (supplies blood to the right side of the head), a left common carotid artery 112 (supplies blood to the left side of the head), a right subclavian artery 114 (supplies blood to the right side of the chest and the right arm), a left subclavian artery 116 (supplies blood to the left side of the chest and the left arm), a brachiocephalic artery 118 (splits into the right common carotid artery 110 and the right subclavian artery 114), a right coronary artery 120 (supplies blood to the right side of the heart), a left coronary artery 122 (supplies blood to the left side of the heart), and a thoracic artery 124 (supplies blood to at least the chest wall).

Referring now to FIG. 2, FIG. 2 illustrates an example of an aorta dissection. An aortic dissection of the aorta 100 occurs when there are one or more entry tears, shown as tears 102, in a layer of the wall of the aorta 100. The tears 102 cause dissection of the aorta 100. Specifically, blood entering the aorta 100 flows into the tears 102 which creates a new flow channel for blood flow between layers of the wall of the aorta, typically referred to as a false lumen. FIG. 2 depicts the false lumen 106 as well as the original flow channel wherein blood flows through the aorta 100, shown as a true lumen 104. Aortic dissections are categorized into type A aortic dissections and type B aortic dissections. Type A aortic dissections involve the ascending aorta 103, while type B aortic dissections do not involve the ascending aorta 103. Aortic dissections often lead to malperfusion, or inadequate (e.g., incorrect) blood flow to the body from the aorta 100. With regard to type A aortic dissections, the false lumen 106 may affect the right coronary artery 120 and/or the left coronary artery 122 and cause cardiac tamponade, where blood accumulates in the heart and severely affects the heart's function. The false lumen 106 may affect the brachiocephalic artery 118, and/or the carotid arteries 110 and 112 and cause stroke, where blood accumulates blocks the cerebral perfusion and severely affects the brain function. With regard to type B aortic dissections, the false lumen 106 causes malperfusion of one or more organs, such that blood flow is restricted to the one or more organs which may result in organ damage and/or ischemia (lack of oxygen).

During aortic dissections, malperfusion occurs due to pressure systems of the aorta 100. Specifically, the systolic/diastolic pressure of the true lumen 104 is about 120/70 mmHg, while the systolic pressure (e.g., pressure when the heart is pumping) is 120 mmHg and the diastolic pressure (e.g., pressure when the heart is filling) is 70 mmHg. In contrast, the systolic/diastolic pressure of the false lumen 106 is about 120/110 mmHg. The diastolic pressure of the false lumen 106 is higher than of the true lumen 104 because there are no organs to perfuse from the false lumen 106. Specifically, since blood cannot flow out of the false lumen 106, the diastolic pressure builds up in the false lumen 106 and cannot reduce. Overall, the mean pressure of the false lumen 106 is greater than the mean pressure of the true lumen 104 which may cause the false lumen 106 to be tense and cause the false lumen 106 to expand into and cause collapse of the true lumen 104. The high pressure and/or stiffness of the false lumen 106 may lead to malperfusion and other complications, such as cardiac tamponade, stroke, myocardial infarction, etc.

Referring to the Figures generally, various embodiments disclosed herein relate to systems and methods for treating aortic dissections, for example for depressurizing a false lumen of an aorta. Specifically, the methods described herein relate to an innovative step of releasing pressure in the false lumen 106 by providing a channel to fluidly couple the false lumen 106 to the venous system. For example, a surgeon or another operator may place a shunt device to allow fluid to flow from the false lumen to the venous system (which is at a far lower pressure than the false lumen 106 and true lumen 104), which may cause the depressurization and collapse of the false lumen 106. The methods as described herein may be used as within various systems (e.g., therapeutic medical systems), such as to treat particular arterial conditions or patient conditions associated with the vascular system. These methods may include other devices, systems, equipment, etc., such as penetration devices to advance within a vessel (e.g., catheters, wires, tubes), imaging equipment (fluoroscopic devices, sonographic devices), devices for providing a flow channel and/or placing a shunt device (e.g., a robotic system for endovascular operations). The teachings herein can thereby treat aortic dissection intravenously, without requiring open surgery of the aorta, which may significantly reduce recovery time and risk of complications as compared to options for treating aortic dissection which involve open surgery or laparoscopic access to the aorta.

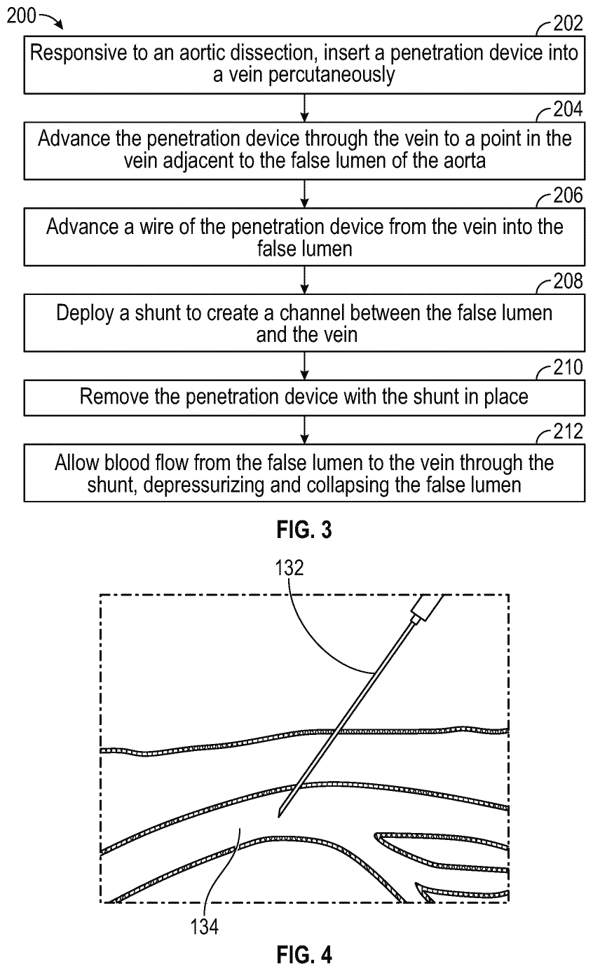

Referring now to FIG. 3, a flowchart of a process 200 for treating an aortic dissection is shown, according to some embodiments. Specifically, process 200 refers to a method for fluidly coupling the false lumen 106 to the venous system, such that a channel is provided between the false lumen 106 and the venous system wherein fluid may flow from the false lumen 106 into the venous system. Additionally, FIGS. 4-10 show various systems, methods, etc. used in process 200. Reference is made thereto to facilitate explanation of process 200. It should be understood that process 200 is not limited to the examples of FIGS. 4-10. Additionally, although FIGS. 4-10 illustrate embodiments of process 200 for treating an aortic dissection, other embodiments are possible for treating dissections relating to other blood vessels, for example carotid artery dissections, vertebral artery dissections, or renal artery dissections.

At step 202, responsive to an aortic dissection occurring, a blood vessel 134 is accessed percutaneously. For example, as shown in FIG. 4, a surgeon or other operator may insert a needle 132 percutaneously to access a blood vessel 134 (e.g., a jugular vein, a femoral vein) of a patient.

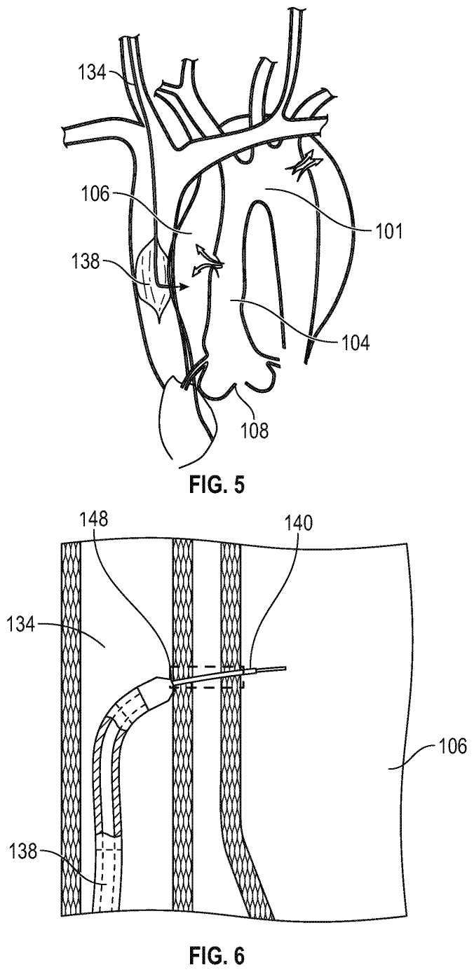

At step 204, a penetration device 138 (e.g., endovascular catheter, guide catheter) is advanced through the blood vessel 134. For example, as shown in FIG. 5, the penetration device is inserted into the blood vessel 134 and advanced until the penetration device 138 reaches a target position within the blood vessel 134, wherein the target position is a position in the blood vessel 134 adjacent to the false lumen 106 of the aorta 100. For example, the penetration device 138 may be manipulated by a surgeon manually and/or using tools for steering of the penetration device 138 to a site in the blood vessel 134 adjacent to the false lumen 106. An imaging technique, such as fluoroscopy or ultrasonography, may be used to facilitate advancing the penetration device 138 and/or determining a position of the penetration device 138 within the vessel 134. The target position in the blood vessel 134 adjacent the false lumen 106 is thereby reached intravenously, without requiring open surgery to access the aorta 100 (or laparoscopic access to the aorta).

In some implementations, the penetration device 138 may include a guide wire hole (e.g., a narrow hole, lumen, central channel, central passage) for inserting a guide wire to the target position. As preparation, a guide wire that has been disposed to pass through the blood vessel 134 to the target position may be inserted through the guide wire hole of the penetration device 138. The guide wire may then be used to advance the penetration device 138 as a whole to the target position. The penetration device 138 is then advanced (e.g., threaded through the needle 132) into the blood vessel 134 toward the target position. In some implementations, the penetration device 138 may be similar to the device as described in greater detail in as described in U.S. application Ser. No. 16/352,259, now U.S. Pat. No. 11,317,922, filed Mar. 13, 2019, the entire disclosure of which is incorporated by reference herein. For example, the penetration device 138 may include an expandable basket or cage that can be expanded at the target position in step 204, for example as shown in FIG. 5.

At step 206, a wire is advanced from the blood vessel 134 into the false lumen 106 of the aorta 100. For example, as shown in FIG. 6, a wire 140 is advanced along the penetration device 138 (e.g., through the penetration device 138 via the guide wire hole) to extend from a distal end of the penetration device 138, shown as distal end 148 (i.e., at the target site). The wire 140 can subsequently penetrate through (pierce through) a wall of the blood vessel 134 adjacent to the false lumen 106. The wire 140 may then penetrate through a wall of the false lumen 106 adjacent to the blood vessel 134, such that the wire 140 extends from the penetration device 138 in the blood vessel 134 to the false lumen 106. For example, the wire 140 may be manipulated manually or via tools for steering of the wire (e.g., magnetic steering, robotic steering), etc. to advance the wire 140 into the false lumen 106. In some embodiments, the wire 140 may be made of a shape-memory material (e.g., nitinol) provided with a shape causing the wire 140 to turn substantially orthogonal to the direction of the penetration device 138 to extend toward and penetrate into the false lumen 106 when advanced beyond the distal end 148. In some embodiments, the penetration device includes a steerable tip configured to be steered (e.g., via manipulation of an input device at an opposite, proximal end of the penetration device outside the patient), to turn toward the walls of the blood vessel 134 and the false lumen 106, thereby aiming the wire 140 toward said walls. The position of the wire 140 in the blood vessel 134 (and the false lumen 106) may be confirmed using an imaging technique, such as fluoroscopy or ultrasonography (e.g., using an intraoperative fluoroscopy system, using an ultrasound device).

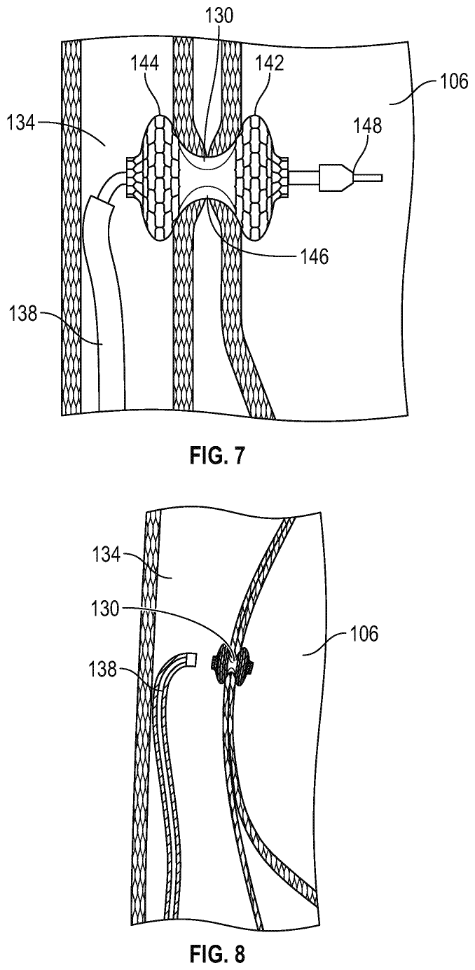

At step 208, a flexible tube or shunt device, shown as shunt 130, is advanced, such that a channel between the false lumen 106 and the blood vessel 134 is provided. For example, as shown in FIG. 7, once at least a part of the wire 140 is disposed in the false lumen 106, the shunt 130 is introduced and advanced. For example, the shunt 130 may be passed (e.g., threaded, advanced, etc.) through the penetration device 138 and over the wire 140 until the shunt 130 reaching a desired position along the wire 140 with the shunt 130 extending between the false lumen 106 and the blood vessel 134 (e.g., a clinically-desired position, a target position along the wire corresponding to a dimension of the shunt to be implanted). At this time, the shunt 130 may be maintained at a compressed state. Responsive to the shunt 130 reaching the desired position along the wire 140, the shunt 130 may then be released or deployed (e.g., by retracing the penetration device 138 and/or wire 140), such that the shunt 130 establishes (via a channel, opening, lumen, etc. through the shunt 13) a fluid connection between the false lumen 106 with the blood vessel 134.

In some embodiments, for example as shown in FIG. 7, the shunt 130 is shown in a deployed configuration. The shunt 130 is configured to transition between an undeployed (e.g., unexpanded) configuration and a deployed (e.g., expanded) configuration. Portions of the shunt 130 may be expanded radially outward in both the false lumen 106 and the blood vessel 134 such that the shunt 130 engages with the inner wall surface of the blood vessel 134 and/or the inner wall surface of the false lumen 106 to provide mechanical engagement which holds the shunt 130 in its deployed configuration. The shunt 130 can include a first radially expanding portion, shown as first expanding portion 142, a second radially expanding portion, shown as second expanding portion 144, and a connecting portion 146 positioned between and coupling the first and second expanding portions 142, 144. The first expanding portion 142 is positioned proximate the distal end 148. The first expanding portion 142 and the second expanding portion 144 may have a same (e.g., substantially same) maximum diameter. A maximum diameter of the connecting portion 146 may be less than the maximum diameters of the first expanding portion 142 and the second expanding portion 144. The first expanding portion 142 is configured to engage with the inner wall surface of the false lumen 106. The second expanding portion 144 is configured to engage with the inner wall surface of the vessel 134. In some cases, in the deployed configuration, the shunt 130 forms a mesh-like peripheral wall. The shunt 130 may include one or more anchoring mechanisms to secure the shunt 130 in place. Thus, the placement of the shunt 130 is completed so as to fluidly couple the false lumen 106 with the blood vessel 134. The shunt 130 may be implanted under guidance of an imaging system, such as fluoroscopy or ultrasonography. A size (e.g., shape) of the shunt 130 may be determined to optimize the flow of fluid from the false lumen 106 to the blood vessel 134. Although the example shunt 130 shown in the Figures defines a generally cylindrical shape, an aspect of the present disclosure is that other shunt designs (e.g., shapes) may be suitable depending on location of the false lumen, position of the stent being placed, size of the false lumen, etc.

At step 210, the penetration device 138 is removed from the blood vessel 134. For example, as shown in FIG. 8, the penetration device 138 is withdrawn from the blood vessel 134 by retracting the penetration device 138 along the blood vessel 134 and out through the same percutaneous entry point created in step 202. Step 210 can be executed responsive to the shunt 130 being implanted. The penetration device 138 as a whole, along with the wire 140 and/or the guide wire, may be withdrawn (e.g., removed) from the blood vessel 134 while the shunt 130 remains implanted across the walls of the blood vessel 134 and the false lumen 106.

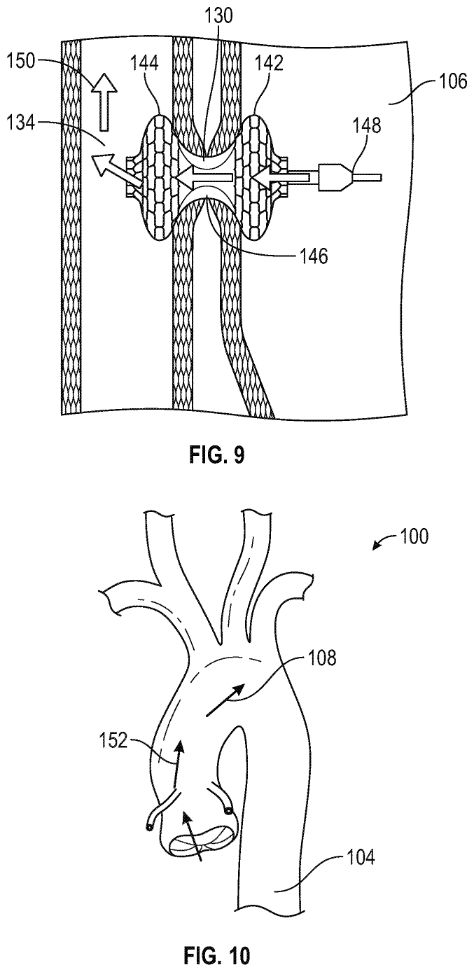

At step 212, the shunt 130 allows a flow of fluid (e.g., blood) through the shunt 130 from the false lumen 106 to the blood vessel 134 through the channel provided by the shunt 130. For example, as shown in FIG. 9, the shunt 130 allows a flow of blood from the false lumen 106 through the shunt 130 to the blood vessel 134, shown by blood flow 150. As described previously, the mean pressure of the false lumen 106 is relatively high (e.g., at approximately 110 mmHg) in comparison to the mean pressure of the blood vessel 134 (e.g., about 0 mmHg, lower than the mean pressure of the false lumen 106), such that fluid will flow from the false lumen 106 to the blood vessel 134 (i.e., the pressure differential will pull or force fluid out of the false lumen 106 and into the blood vessel 134). The channel as provided by the shunt 130 diverts the blood in the false lumen 106 to another area of the body by allowing the blood to exit the false lumen 106 to the venous system. Prior to the channel being provided, there is no such outlet for fluid to flow from the false lumen 106 once the fluid enters the false lumen 106, resulting in the false lumen 106 having a high diastolic pressure (e.g., approximately 110 mmHg). Providing a channel between the false lumen 106 and the blood vessel 134, allows blood to exit the false lumen 106, reducing the pressure of the false lumen 106.

The depressurization of the false lumen 106 and flow of fluid out of the false lumen 106 may advantageously also cause the collapse of the false lumen 106. In particular, allowing depressurization of the false lumen 106 via the stent in step 212 can cause the pressure in the false lumen 106 to drop below the pressure in the true lumen 104, at which point said pressure in the true lumen 104 can start to push against and cause collapse of (reduction in size of shrinking of, closure of, etc.) the false lumen 106.

Process 200 thereby leverages the pressure differential between the true lumen 104, the false lumen 106, and the blood vessel 134 to cause fluid to flow out of the false lumen 106 and to cause the false lumen 106 to collapse (e.g., to cause the torn partial wall of the aorta to return to its pre-dissection position abutting a remainder of the wall of the aorta). The aortic dissection is thereby treated. Over time, as shown in FIG. 10, with the false lumen 106 collapsed in a manner caused by process 200, the tears 102 may heal and thereby resolving the patient's aortic dissection. The collapse of the false lumen 106 allows fluid (blood) entering and flowing through the aorta 100 to flow through the true lumen 104, shown as flow pattern 152 (e.g., to cause the flow of blood through the aorta 100 to return to its pre-dissection flow pattern).

In some implementations, responsive to decompression of the false lumen 106 (e.g., remodeling) and/or other tissue changes reflecting at clinical improvement of the aortic dissection, the shunt 130 may be removed to complete the surgical procedure. For example, the shunt 130 may be accessed intravenously (along a similar path and technique as used to place the shunt 130) and unanchored from its position and withdrawn through the vessel 134. Removal of the shunt 130 may cause the wall of vessel 134 to quickly seal, heal, collapse, etc. any hole left by removal of the shunt 130. In some other implementations, the surgical procedure may end with the shunt 130 still implanted in the vessel 134, such that the shunt 130 is permanent; in such scenarios, the shunt 130 may provide for continued mitigation of future aortic dissections. In some implementations, the shunt 130 may be left in place for a desired amount of time (e.g., a clinically-desired amount of time, an amount of time for the false lumen 106 to collapse) before being surgically removed.

For example, in some embodiments, the shunt 130 remains connected to the penetration device 138 and/or the wire 140, a portion of which can remain in the vein to facilitate retrieval of the shunt 130 after decompression of the false lumen 106 (e.g., and/or after other time for healing of tissue, in some scenarios). For example, in some scenarios, after a time in the range of about one to two weeks, the shunt 130 may be retrieved (removed, explanted, etc.) following a reverse procedure as described above for implantation of the shunt 130, for example by pulling the shunt 130 back into a catheter (e.g., into the penetration device 138, resheathed), thereby collapsing the shunt 130 into an undeployed state such that it can be removed from the patient.

In some implementations, the methods as described herein may be similar to the method for reaching a vessel using a device as described in greater detail in as described in U.S. application No. Ser. No. 16/352,259, now U.S. Pat. No. 11,317,922, filed Mar. 13, 2019.

In some embodiments, the device used herein is as described in Japanese Patent Publication No. JP 2025164688A published Oct. 30, 2025, the entire disclosure of which is incorporated by reference herein.

The systems and methods described herein for treating aortic dissections provide numerous advantages over traditional methods. For example, current methods to treat type A aortic dissections may include opening a chest surgically and using a cardiopulmonary pump to circulate blood during the procedure. A surgeon may then replace the ascending aorta 103 (and/or the aortic valve 108, the right coronary artery 120, and/or the left coronary artery 122 as needed), such as with a graft (e.g., stent graft, synthetic graft, frozen elephant trunk, etc.). Such procedures require open surgery which is less preferable compared to minimally invasive procedures. By way of another example, current methods to treat type B aortic dissections may include placing a stent graft to seal the primary entry tear of the aorta 100. The stent graft may be extended to lower sections of the aorta 100 (e.g., near the thoracic artery 124) to expand the true lumen 104. Often, a metal stent may be placed distally at one or more visceral arteries. Such procedures allow the false lumen 106 to remain, and the false lumen 106 may require repair later on. Further, sealing of the entry tear 102 (e.g., by a stent graft) does not reverse the high pressure of the false lumen 106. Current surgical procedures that involve stent grafts to treat aortic dissections can cover a primary (e.g., main) entry tear, but may still leave one or more secondary (e.g., smaller) entry tears. The one or more secondary entry tears may still cause issues if left untreated. Advantageously, the systems and methods as described herein enable surgeons to perform endovascular shunt placement to treat aortic dissections, which is a minimally invasive procedure. Additionally, the systems and methods as described herein result in remodeling, such that the false lumen is collapsed. This advantageously may lead to reduced recovery time, lower risk of complications, less pain, and/or overall improved outcomes for patients, as compared to other processes for treating aortic dissection.

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, use of materials, colors, orientations, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and are considered to be within the scope of the disclosure.

1. A surgical method for treating aortic dissection, comprising:
advancing a penetration device along a vein to a position in the vein adjacent a false lumen of an aorta;
advancing a wire at a distal end of the penetration device from the vein into the false lumen of the aorta;
establishing a fluid connection between the vein and the false lumen by advancing a shunt device along the wire to a position connecting the vein with the false lumen without extending into a true lumen of the aorta; and
causing, by the fluid connection, collapse of the false lumen by depressurizing the false lumen relative to the true lumen, thereby treating the aortic dissection.

2. The surgical method of claim 1, wherein the fluid connection allows fluid to flow from the false lumen to the vein.

3. The surgical method of claim 2, further comprising determining a size of the shunt device to optimize a flow of the fluid from the false lumen to the vein.

4. The surgical method of claim 1, wherein the vein is one of (a) a jugular vein or (b) a femoral vein.

5. The surgical method of claim 1, further comprising, responsive to establishing the fluid connection between the vein and the false lumen, removing the penetration device and the wire from the vein.

6. The surgical method of claim 1, further comprising, responsive to the depressurization and the collapse of the false lumen, removing the shunt device.

7. The surgical method of claim 1, wherein establishing the fluid connection between the vein and the false lumen further comprises transitioning the shunt device from an undeployed configuration to a deployed configuration.

8. The surgical method of claim 7, wherein the shunt device includes a first radially expanding portion, a second radially expanding portion, and a connecting portion positioned between the first and second radially expanding portions.

9. The surgical method of claim 8, wherein a diameter of each of the first and second radially expanding portions is greater than a diameter of the connecting portion in the deployed configuration.

10. A surgical method for treating aortic dissection, comprising:
advancing a device along a wire to be positioned between a vein and a false lumen of an aorta;
expanding the device to establish a fluid connection between the vein and the false lumen;
causing, by the fluid connection, depressurization of the false lumen thereby treating the aortic dissection; and
removing the device responsive to the depressurization of the false lumen.

11. The surgical method of claim 10, wherein the fluid connection allows fluid to flow from the false lumen to the vein.

12. The surgical method of claim 10, wherein the wire extends from a distal end of a penetration device, further comprising, before advancing the device along the wire, advancing the penetration device along the vein to a position in the vein adjacent the false lumen.

13. The surgical method of claim 12, further comprising, responsive to expanding the device, removing the penetration device from the vein.

14. The surgical method of claim 12, further comprising, responsive to establishing the fluid connection between the vein and the false lumen, removing the penetration device.

15. The surgical method of claim 10, wherein the vein is one of (a) a jugular vein or (b) a femoral vein.

16. The surgical method of claim 10, wherein the device includes a first radially expanding portion, a second radially expanding portion, and a connecting portion positioned between the first and second radially expanding portions, wherein a diameter of each of the first and second radially expanding portions is greater than a diameter of the connecting portion.

17. A system for treating aortic dissection, in which a torn layer of an aortic wall separates a false lumen from a true lumen of an aorta, the system comprising:
a penetration device configured to advance along a vein to a position in the vein adjacent a false lumen of an aorta and including a wire at a distal end thereof, the wire configured to extend into the false lumen; and
a shunt device configured to advance along the wire and establish a fluid connection between the vein and the false lumen in a deployed configuration without extending through the torn layer of the aortic wall, the shunt device comprising a first radially expanding portion, a second radially expanding portion, and a connecting portion positioned between the first and second radially expanding portions,
wherein the fluid connection causes collapse of the false lumen by creating a pressure differential across the torn layer between the false lumen and the true lumen, thereby treating the aortic dissection.

18. The system of claim 17, wherein the fluid connection allows fluid to flow from the false lumen to the vein.

19. The system of claim 17, wherein a diameter of each of the first and second radially expanding portions is greater than a diameter of the connecting portion in the deployed configuration.

20. The system of claim 17, wherein the shunt device is at least one of temporary or removable.

21. A method for treating a dissected aorta in which a torn layer of an aortic wall separates a false lumen from a true lumen of an aorta, the method comprising:
surgically establishing a fluid connection between the false lumen of a dissected aorta and a vein adjacent to the false lumen; and
causing, by the fluid connection, depressurization of the false lumen relative to the true lumen such that a pressure differential between the true lumen and the false lumen forces the torn layer toward a position abutting a remainder of the aortic wall, thereby treating the dissected aorta.
